SEQUENCE LISTING (10 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 30 aa, Artificial Sequence
  SYNTHETIC CONSTRUCT
FVNQHLCGSHLVEALYLVCGERGFFYTKPT

SEQ ID NO: 5 - protein, 59 aa, Artificial Sequence
  SYNTHETIC CONSTRUCT
FVNQHLCGSHLVEALYLVCGERGFFYTPRTEEGSRRSRGIVEQCCRSICSLYQLENYCG

SEQ ID NO: 6 - protein, 57 aa, Artificial Sequence
  SYNTHETIC CONSTRUCT
FVNQHLCGSHLVEALYLVCGERGFFYTPETEEGPRRGIVEQCCTSICSLEQLENYCN

SEQ ID NO: 7 - protein, 57 aa, Artificial Sequence
  SYNTHETIC CONSTRUCT
FVNQHLCGSHLVEALYLVCGERGFFYTDPTEAAAAAGIVEQCCTSICSLEQLENYCN

SEQ ID NO: 8 - protein, 57 aa, Artificial Sequence
  SYNTHETIC CONSTRUCT
FVNQHLCGSHLVEALYLVCGERGFFYTDPTEAAARAGIVEQCCTSICSLEQLENYCN

SEQ ID NO: 9 - protein, 62 aa, Artificial Sequence
  SYNTHETIC CONSTRUCT
  MISC_FEATURE (14)..(14): Xaa is Ala or Gln
  MISC_FEATURE (29)..(29): Xaa is Arg or Lys
  MISC_FEATURE (31)..(41): Xaa is any 5 to 11 of amino acid
  MISC_FEATURE (49)..(49): Xaa is Lys, Arg or His
  MISC_FEATURE (62)..(62): Xaa is Gly, Ala, Ser, or Gln
FVNQHLCGSHLVEXLYLVCGERGFFYTPXTXXXXXXXXXXXGIVEQCCXSICSLYQLENYCX

SEQ ID NO: 10 - protein, 62 aa, Artificial Sequence
  SYNTHETIC CONSTRUCT
  MISC_FEATURE (28)..(28): Xaa is Lys, Pro or Glu
  misc_feature (29)..(29): Xaa can be any naturally occurring amino acid
  MISC_FEATURE (31)..(41): Xaa is any 5 to 11 of amino acid
  MISC_FEATURE (49)..(49): Xaa is any amino acid other than Leu, Ile, or Val
  MISC_FEATURE (55)..(55): Xaa is Glu, Ala or Tyr
  MISC_FEATURE (62)..(62): Xaa is Asn, Gly, Ala, Ser or Gln
FVNQHLCGSHLVEALYLVCGERGFFYTXXTXXXXXXXXXXXGIVEQCCXSICSLXQLENYCN

(19) United States
(12) Patent Application Publication
(10) Pub. No.: US 2022/0280614 A1
(43) Pub. Date: Sep. 8, 2022

(54) Premixed Ultra-Stable Single-Chain Insulin Analogue Formulations

(71) Applicant: CASE WESTERN RESERVE UNIVERSITY, Cleveland, OH (US)

(72) Inventors: Michael A. WEISS, Indianapolis, IN (US); Faramarz ISMAIL-BEIGI, Shaker Heights, OH (US)

(21) Appl. No.: 17/632,193

(22) PCT Filed: Aug. 3, 2020
(86) PCT No.: PCT/US2020/044788

§ 371 Date: Feb. 1, 2022

Related U.S. Application Data
(60) Provisional application No. 62/882,012, filed on Aug. 2, 2019.

(51) Int. Cl. A61K 38/28; A61P 3/10; C07K 14/62; A61K 47/02

(52) U.S. Cl. CPC A61K 38/28; A61P 3/10; C07K 14/62; A61K 47/02

(57) ABSTRACT

A premixed acidic solution contains two single-chain insulin analogues. One has isoelectric point between 6.5 and 8.0 (SCI-A) and the other has isoelectric point between 4.5 and 6.0 (SCI-B), such that biphasic basal and prandial insulin activity is provided on subcutaneous injection. Each protein is an analogue of a mammalian insulin, such as human insulin, with insertion of engineered C domain of length 5-11 residues; the respective C domains of SCI-A and SCI-B may be different. SCI-A contains a Glycine, Alanine, Serine or Glutamine substitution at position A21 and may contain a basic residue at position A8 and Glutamine at position B13. SCI-B may contain a non-beta-branched substitution at position A8, either Alanine or Glutamic Acid at position A14, and substitutions at positions B28 and/or B29 to confer rapid action. A method of treating a patient comprises administering the insulin analogue or a physiologically acceptable salt thereof to a patient.

### CROSS REFERENCE TO RELATED APPLICATIONS

This is a national stage application of PCT/US2020/044788, filed on Aug. 3, 2020, which claims benefit of pending U.S. Provisional Application No. 62/822,012 filed on Aug. 2, 2019, the contents of which are incorporated by reference herein.

### STATEMENT REGARDING FEDERALLY SPONSORED RESEARCH OR DEVELOPMENT

This invention was made with government support under grant numbers DK040949 and DK074176 awarded by the National Institutes of Health. The government has certain rights in the invention.

### BACKGROUND OF THE INVENTION

This invention relates to mixtures of polypeptide hormone analogues that exhibit enhanced pharmaceutical properties, such as increased thermodynamic stability, augmented resistance to thermal fibrillation above room temperature, decreased mitogenicity, and/or altered pharmacokinetic and pharmacodynamic properties, i.e., conferring a biphasic time course of action relative to (a) a fast-acting component similar to soluble formulations of the corresponding prandial or wild-type human hormone and (b) a prolonged component similar to either a soluble basal insulin analogue or microcrystalline NPH suspension of wild-type insulin (or an insulin analogue). The insulin analogue co-formulations of the present invention contain soluble proteins and exhibit prolonged shelf life at or above room temperature relative to wild-type (WT) insulin formulations, relative to soluble formulations of current insulin analogues in clinical use, and relative to their respective microcrystalline NPH suspensions.

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often confer multiple biological activities. A benefit of non-standard proteins would be to achieve selective activity, such as decreased binding to homologous cellular receptors associated with an unintended and unfavorable side effect, such as promotion of the growth of cancer cells. Yet another example of a societal benefit would be augmented resistance to degradation at or above room temperature, facilitating transport, distribution, and use.

An example of a therapeutic protein is provided by insulin. Wild-type human insulin and insulin molecules encoded in the genomes of other mammals bind to insulin receptors is multiple organs and diverse types of cells, irrespective of the receptor isoform generated by alternative modes of RNA splicing or by alternative patterns of post-translational glycosylation. Wild-type insulin also binds with lower affinity to the homologous Type 1 insulin-like growth factor receptor (IGF-1R).

An example of a further medical benefit would be optimization of the pharmacokinetic (PK) properties of a soluble formulation such that the time course of insulin action has two phases, a rapid phase and a delayed phase (FIG. 1). Such a combination of rapid and delayed phases is known in the art to be conferred by mixtures of a solution of zinc insulin analogue hexamers (as provided by, but not limited to, insulin lispro and insulin aspart) with a micro-crystalline suspension of the same analogue prepared in combination with zinc ions and protamines or protamine-related basic peptides; the latter component is designated in the art as Neutral Protamine Hagedorn (NPH) micro-crystalline suspensions. Pre-mixed insulin products known in the art contain varying ratios of these two components, such as 25% soluble phase and 75% micro-crystalline phase, 30% soluble phase and 70% micro-crystalline phase, or 50% of each phase. Such pre-mixed products are widely used by patients with diabetes mellitus in the developing world due to their ease of use with reduction in the number of subcutaneous injections per day relative to the separate administration of a prandial (rapid-acting) insulin formulation (or prandial insulin analogue formulation) and of a NPH micro-crystalline suspension of wild-type insulin or insulin analogue. The simplification of insulin regimens of insulin provided by pre-mixed biphasic insulin products has also proven of benefit to patients with diabetes mellitus in affluent societies (i) for whom treatment solely by prandial insulin analogue formulations leads to suboptimal glycemic control or excessive weight gain, (ii) for whom treatment solely by NPH insulin products or basal insulin analog formulations leads to suboptimal glycemic control due to upward excursions in the blood glucose concentration within three hours after a meal, or (iii) patients of the above two classes for whom addition of an oral agent (such as metformin) does not result in satisfactory glycemic control.

Existing biphasic insulin products require a complex and costly method of manufacture due to the post-fermentation and post-purification steps needed to grow NPH micro-crystals. Further, such products suffer from an intrinsic susceptibility of both the soluble and micro-crystalline components to physical and chemical degradation above room temperature. The biphasic pharmacokinetic properties of these pre-mixed products may change with storage of the vials above room temperature due to interchange of insulin molecules between the soluble and micro-crystalline phases. Finally, the use of micro-crystalline suspensions can be associated with uncertainties in dosing as the number of micro-crystals drawn into a syringe can vary from withdrawal to withdrawal even from the same vial.

In light of the above disadvantages of existing biphasic insulin products, the therapeutic and societal benefits of biphasic insulin formulations would be enhanced by the engineering of mixtures of two ultra-stable insulin analogues—one fast acting and the other slowly absorbed from a joint subcutaneous depot—thereby conferring a biphasic pattern of insulin action. SCIs provide a class of ultra-stable insulin analogues whose isoelectric points may be engineered to modulate PK properties on subcutaneous injection. Additional benefits would accrue if the constituent insulin analogues were simpler and less costly to formulate (i.e., by avoiding the requirement for micro-crystallization) and/or if the novel soluble insulin analogue were more refractory than wild-type insulin to chemical or physical degradation at or above room temperature. Such resistance to degradation above room temperature would be expected to facilitate use in regions of the developing world where electricity and refrigeration are not consistently available. The challenge posed by such degradation is deepened by the pending pandemic of diabetes mellitus in Africa and Asia. Because fibrillation poses the major route of degradation above room temperature, the design of fibrillation-resistant formulations may enhance the safety and efficacy of insulin replacement therapy in such challenged regions. Still additional therapeutic and societal benefit would accrue if each of the two-component single-chain insulins (SCIs) should exhibit (in assays developed to monitor insulin-stimulated proliferation of human cancer cell lines) reduced mitogenicity and similarly if premixed solutions of the two SCIs should exhibit reduced mitogenicity.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure.

Insulin is a small globular protein that plays a central role in metabolism in vertebrates. Insulin contains two chains, an A chain, containing 21 residues, and a B chain containing 30 residues. The hormone is stored in the pancreatic β-cell as a Zn2+-stabilized hexamer, but functions as a Zn2+-free monomer in the bloodstream. Insulin is the product of a single-chain precursor, proinsulin, in which a connecting region (35 residues) links the C-terminal residue of B chain (residue B30) to the N-terminal residue of the A chain. A variety of evidence indicates that it consists of an insulin-like core and disordered connecting peptide. Formation of three specific disulfide bridges (A6—A11, A7—B7, and A20—B19) is thought to be coupled to oxidative folding of proinsulin in the rough endoplasmic reticulum (ER). Proinsulin assembles to form soluble Zn2+-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). Individual residues are indicated by the identity of the amino acid (typically using a standard three-letter code), the chain and sequence position (typically as a superscript).

Fibrillation, which is a serious concern in the manufacture, storage and use of insulin and insulin analogues for the treatment of diabetes mellitus, is enhanced with higher temperature, lower pH, agitation, or the presence of urea, guanidine, ethanol co-solvent, or hydrophobic surfaces. Current US drug regulations demand that insulin be discarded if fibrillation occurs at a level of one percent or more. Because fibrillation is enhanced at higher temperatures, patients with diabetes mellitus optimally must keep insulin refrigerated prior to use. Insulin exhibits an increase in degradation rate of 10-fold or more for each 10° C. increment in temperature above 25° C.; accordingly, guidelines call for storage at temperatures <30° C. and preferably with refrigeration. The NPH micro-crystalline component of existing biphasic insulin product is susceptible to fibrillation above room temperature as well as to a distinctive mode of chemical degradation due to proteolytic cleavage within the A-chain; such cleavage inactivates the insulin or insulin analogue. The resistance of similar single-chain insulin analogs to fibrillation at or above room temperature has been previously demonstrated by prolonged heating of insulin analogue formulations with gentle agitation, followed by biological testing in diabetic rats (see FIGS. 6A and 6B).

The above cleavage of the insulin A-chain in NPH micro-crystals is representative of a process involving the breakage of chemical bonds. Such breakage can lead to loss or rearrangement of atoms within the insulin molecule or the formation of chemical bonds between different insulin molecules, leading to formation of polymers. Whereas cleavage of the A-chain in NPH micro-crystals is thought to occur on the surface of the folded state, other changes in chemical bonds are mediated in the unfolded state of the protein or in partially unfolded forms of the protein, and so modifications of insulin that augment its thermodynamic stability also are likely to delay or prevent chemical degradation. It is therefore a desirable property of an insulin analogue that its free energy of denaturation (as typically measured by circular dichroism at a helix-sensitive wavelength as a function of the concentration of a chemical denaturant) should be equal to or greater than that of wild-type insulin or equal to or greater than that of a prandial (rapid-acting) insulin analogue in current clinical use.

Insulin is also susceptible to physical degradation. The present theory of protein fibrillation posits that the mechanism of fibrillation proceeds via a partially folded intermediate state, which in turn aggregates to form an amyloidogenic nucleus. In this theory, it is possible that amino-acid substitutions that stabilize the native state may or may not stabilize the partially folded intermediate state and may or may not increase (or decrease) the free-energy barrier between the native state and the intermediate state. Therefore, the current theory indicates that the tendency of a given amino-acid substitution in the two-chain insulin molecule to increase or decrease the risk of fibrillation is highly unpredictable. Models of the structure of the insulin molecule envisage near-complete unfolding of the three alpha-helices (as seen in the native state) with parallel arrangements of beta-sheets forming successive stacking of B-chains and successive stacking of A-chains; native disulfide pairing between chains and within the A-chain is retained. Such parallel cross-beta sheets require substantial separation between the N-terminus of the A-chain and C-terminus of the B-chain (>30 Å), termini ordinarily in close proximity in the native state of the insulin monomer (<10 ∈). Marked resistance to fibrillation of single-chain insulin analogues with foreshortened C-domains is thought to reflect a topological incompatibility between the splayed structure of parallel cross-beta sheets in an insulin protofilament and the structure of a single-chain insulin analogue with native disulfide pairing in which the foreshortened C-domain constrains the distance between the N-terminus of the A-chain and C-terminus of the B-chain to be unfavorable in a protofilament. A ribbon model of a single-chain insulin analogue is shown in FIG. 2; a space-filling model of the insulin moiety is shown in FIG. 3 to highlight the role of the engineered connecting domain (C domain; stick representation in FIG. 3).

### SUMMARY OF THE INVENTION

The present invention was motivated by the medical and societal needs to engineer a biphasic ultra-stable insulin analogue solution. These needs will be addressed via a premixed solution of two SCIs in an acidic formulation where one protein has an isoelectric point of between 4.5 and 6.0 where the other has an isoelectric point between 6.5 and 7.5. The premixed formulation is intended for once-a-day or twice-a-day injection, i.e., on a schedule similar to that of current pre-mixed regular-NPH biphasic insulin products; it may also be used once a day before the largest meal of the day in cultures in which the great majority of calories are consumed at a single specific meal per day (e.g., at breakfast or at dinner).

More particularly, this invention relates to a mixture of insulin analogues consisting of a single polypeptide chain that (i) contains a foreshortened connecting (C) domains between A and B domains with acidic residues at the first and second positions, (ii) contains an amino-acid substitution at position A8 such that one single-chain insulin analog (SCI) has an isoelectric point in the range 4.5-6.0 (designated herein the “rapid-acting SCI” or “SCI-B”) whereas the other has a shifted isoelectric point in the range 6.5-7.5 (designated herein the “pI-shifted SCI” or “SCI-A”). Pertinent to the present invention is the invention of novel foreshortened C domains of length 5-11 residues, which may comprise an N-terminal acidic motif and a C-terminal basic motif, in place of the 36-residue wild-type C domain characteristic of human proinsulin and in combination with amino-acid substitutions at positions A8 and A14 of the A chain and optionally at positions B28 and B29 of the B chain.

These analogues may optionally comprise stabilizing substitutions at positions A8 and/or A14 and may optionally comprise substitutions at positions B28 and/or B29 known in the art to confer rapid action. Also, the C domain of the pI-shifted SCI consist of an N-terminal acidic element and a C-terminal segment basic element; these analogues may contain substitutions at positions B14, B29 and A8 chosen to optimize pI and promote self-assembly at neutral pH. Because co-formulation under acidic conditions is envisaged, both the rapid-acting SCI and the pI-shifted SCI may contain a substitution of AsnA21 to avoid its deamination and chemical degradation under these conditions.

It is, therefore, an aspect of the present invention to provide a pair of single-chain insulin analogues (designated SCI-A and SCI-B) that, when premixed in a single acidic formulation, provide biphasic pharmacokinetic and pharmacodynamics properties on subcutaneous injection. SCI-A and SCI-B represent distinct classes of single-chain insulin analogues distinguishable by their respective isoelective points: pI 6.5-8.0 (SCI-A) versus pI 4.5-6.0 (SCI-B).

In general, the present invention provides pharmaceutical formulation comprising a premixed clear mixture of a first single-chain insulin analogue and a second single-chain insulin analogue, wherein the first and second single-chain insulin analogues comprise the structure B-C-A, where B is an insulin B-chain polypeptide, where A is an insulin A-chain polypeptide and wherein C is a connecting polypeptide of 5-11 amino acids in length, wherein the first insulin analogue (SCI-A) has an isoelectric point between 6.5 and 8.0 and where the second insulin analogue (SCI-B) has an isoelectric point of between 4.5 and 6.0.

SCI-A has an isoelectric point that is shifted (relative to WT insulin) into the range 6.5-8.0 due to an increased net number of positive charges. SCI-B has an isoelectric point that is similar to that of WT insulin (approx. 4.5-6.0) and may be lower due to an increased net number of negative charges. Respectively designated SCI-A and SCI-B, the former provides the basal component of a biphasic formulation whereas the latter provides the rapid-acting component. The pH of the formulation may be between pH 2.9 and 4.1.

The A-chain (or A-domain) polypeptides of SCI-A and/or SCI-B may comprise a substitution at the position corresponding to A21 of wild type insulin (the C-terminus of the SCIs) selected from Gly, Ala, Gln or Ser. The A-domain of SCI-A may also comprise a substitution of a basic amino acid at the position corresponding to position A8 of wild type insulin. Basic amino acids include Lysine, Arginine and Histidine, and also the non-standard amino acid Ornithine. The B-chain polypeptide (or B-domain) of SCI-A may comprise an Arg substitution at the position corresponding to B29 of wild type insulin. In addition or in the alternative, SCI-A may have a Gln substitution at the position corresponding to position B13 relative to wild type insulin.

The A-domain of SCI-B may comprise a substitution at the position corresponding to A21 of wild type insulin (the C-terminus of the SCIs) selected from Gly, Ala, Gln or Ser as mentioned above, and may additionally comprise a Glu, Ala Gln, His, Lys or Arg substitution at the position corresponding to A8 of wild type insulin, a substitution at the position corresponding to A14 of wild type insulin selected from Glu or Ala, or both. Alternatively, the substitution at A8 may be any non-Beta-branched amino acid, that is, an amino acid other than Leu, Ile, or Val.

### BRIEF DESCRIPTION OF THE SEVERAL VIEWS OF THE DRAWINGS

FIG. 1 is a schematic representation of the goal of biphasic insulin products. Original implementations used wild-type insulin (regular and NPH) whereas current products employ prandial insulin analogs. This figure was obtained from R. Beaser & S. Braunstein, MedScape Multispeciality (Education/CME section; 2009) (http://www.medscape.org/viewarticle/708784).

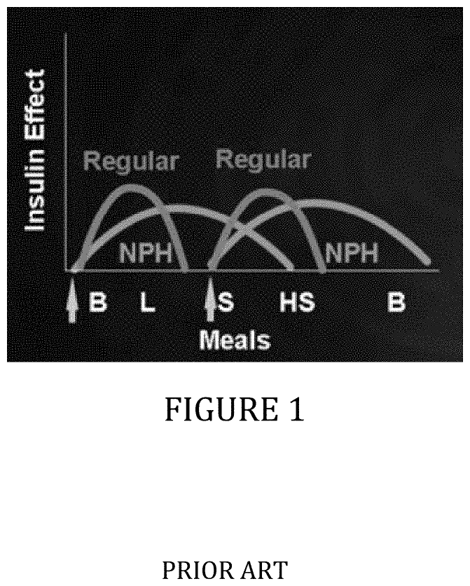

FIG. 2 is a ribbon model of a 57-residue SCI. α-Helices (α₁, α₂, α₃) and the B24-B28 β-strand (arrow) are shown as indicated. The three disulfide bridges (cystines) are shown with asterisks. The C-domain represented is a six amino acid polypeptide.

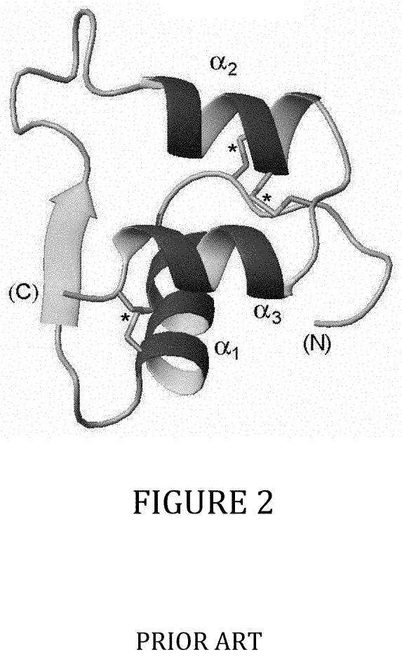

FIG. 3 is a representation of the molecular structure of the 57-residue SCI platform. The six-residue C-domain (sticks) provides a tether between A- and B domains (space-filling representation). The surface shows electrostatic (gray; and white, neutral). This image is based upon NMR studies.

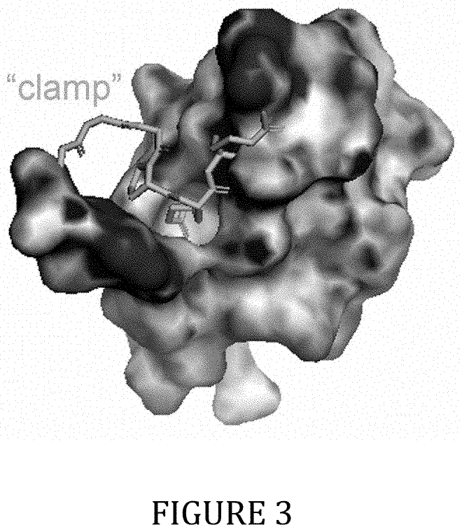

FIG. 4 is a schematic representation of prior biphasic insulin products containing a soluble phase (single zinc hexamers) and an insoluble phase (associated NPH microcrystalline suspension).

FIG. 5A is a graph showing Arterial Plasma Glucose over time for dogs based on a euglycemic clamp following subcutaneous injection of Humalog® U-100 (Eli Lilly and Co., Indianapolis, Ind.; light gray; rapid-acting) and Humalog® Mix75/25™ (Eli Lilly and Co., Indianapolis, Ind.; dark gray; biphasic) as performed by Prof. Alan Cherrington and coworkers at Vanderbilt University.

FIG. 5B is a graph showing the peripheral glucose infusion rate over time for dogs following subcutaneous injection of Humalog® U-100 (Eli Lilly and Co., Indianapolis, Ind.; light gray; rapid-acting) and Humalog® Mix75/25™ (Eli Lilly and Co., Indianapolis, Ind.; dark gray; biphasic) as performed by Prof. Alan Cherrington and coworkers at Vanderbilt University.

FIG. 6A is a graph of the fraction of initial arterial plasma glucose over time for diabetic rats (time 0 blood glucose of 410±20 mg/dl) injected subcutaneously with 1 unit of the indicated analogue/300 g body weight, using fresh analogue samples. Fresh samples: (♦) SCI-57DP (labeled “SCI-2”), (A) Insulin glargine (labelled “glargine”), ( [symbol] ) insulin lispro (labelled “lispro”), and (●) diluent.

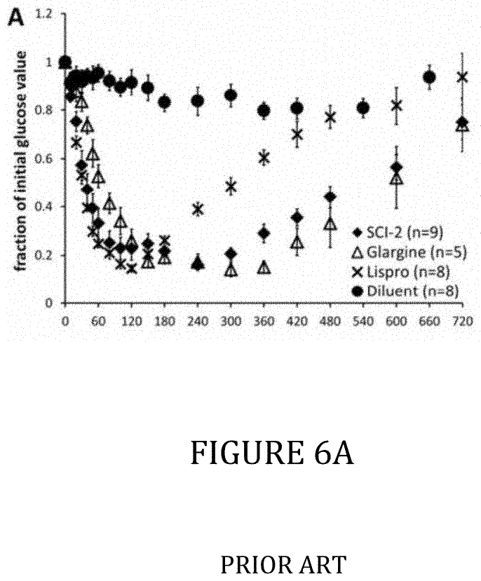

FIG. 6B is a graph of the fraction of initial arterial plasma glucose over time for diabetic rats (time 0 blood glucose of 410±20 mg/dl) injected subcutaneously with 1 unit of the indicated analogue/300 g body weight, using: (♦) fresh SCI-57DP (SCI-2); (□) SCI-57DP (SCI-2) agitated at 45° C. for 57 days and (◯) Insulin glargine (glargine) agitated at 45° C. for 11 days.

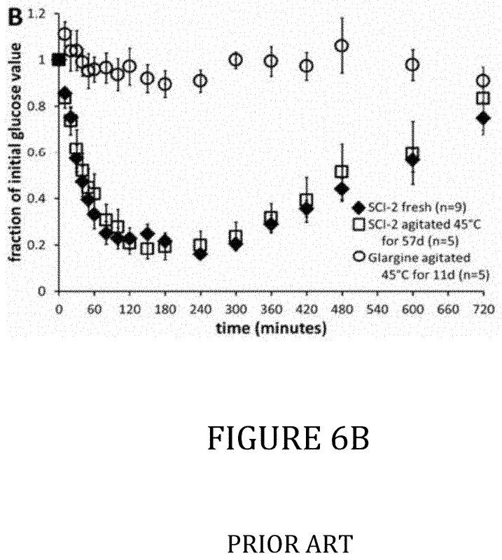

FIG. 7 is a graph showing blood glucose levels over time for diabetic rats (time 0 blood glucose of 375±20 mg/dl) injected subcutaneously with indicated analogue/300 g body weight. ( [symbol] ) diluent control; (●) an SCI-A with C domain EEGSRRSR and insulin-moiety substitutions ArgA8, GlyA21 and ArgB29 (SEQ ID NO: 5); (▪) Humalog® (Eli Lilly and Co., Indianapolis, Ind., insulin lispro) as formulated by Eli Lilly and Co. at neutral pH; and (♦) premixed clear solution of insulin lispro and the SCI-A as formulated in soluble form (similar to Lantus® insulin glargine; Sanofi, Paris, FR) in an unbuffered solution at pH 4.0. Respective doses: 0.75 units SCI-A, 0.25 units Humalog® (Eli Lilly and Co., Indianapolis, Ind.); and premixture of 0.75 units SCI-A and 0.25 units insulin lispro.

FIG. 8 is a graph showing blood glucose levels over time for diabetic rats (time 0 blood glucose of 350-400 [±20 mg/dl]) injected subcutaneously with 1 unit of the indicated analog/300 g body weight. ( [symbol] ) diluent control; (●) an SCI-A (SEQ ID NO: 5); (●) an example of an SCI-B (SEQ ID NO: 6) as formulated at neutral pH; and (♦) equimolar premixed clear solution of this SCI-A and this specific SCI-B as formulated in soluble form in an unbuffered solution at pH 4.0. Respective doses: 20 μg of each protein per 300 gram rat (singly); 20 μg and 20 μg in the case of the premixed solution to a total [SCI-A+SCI-B] combined dose of 40 μg.

FIG. 9 is a graph showing blood glucose levels over time for diabetic rats (time 0 blood glucose of 400±20 mg/dl) injected subcutaneously with indicated analogue/300 g body weight. ( [symbol] ) diluent control; (●) an SCI-A (SEQ ID NO: 5); (▴) an SCI-B (SEQ ID NO: 7) as formulated at pH 4; and (♦) premixed clear solution of insulin SCI-A and SCI-B as formulated in soluble form in an unbuffered solution at pH 4.0. Respective doses: 0.75 units SCI-A, 0.25 units SCI-B; and premixture of 0.75 units SCI-A and 0.25 units SCI-B.

FIG. 10 is a graph showing blood glucose levels over time for diabetic rats (time 0 blood glucose of 385±20 mg/dl) injected subcutaneously with indicated analogue/300 g body weight. ( [symbol] ) diluent control; (●) an SCI-A (SEQ ID NO: 5); ( [symbol] ) an SCI-B (SEQ ID NO: 8) as formulated at pH 4; and (♦) premixed clear solution of insulin SCI-A and SCI-B as formulated in soluble form in an unbuffered solution at pH 4.0. Respective doses: 0.75 units SCI-A, 0.25 units SCI-B; and premixture of 0.75 units SCI-A and 0.25 units SCI-B.

### DETAILED DESCRIPTION OF THE INVENTION

The present invention is directed toward a premixed solution of at least two single-chain insulin analogues in an acidic solution. One insulin analogue is a single polypeptide/protein (designated SCI-A to represent a class of single-chain insulin analogues) that has an isoelectric point between 6.5 and 8.0. It provides rapid duration of action on subcutaneous injection. The other insulin analogue is a single polypeptide/protein (designated SCI-B to represent a distinct class of single-chain insulin analogues) that has an isoelectric point between 4.5 and 6.0. It provides protracted duration of action on subcutaneous injection. Together, a premixed solution (formulated in an unbuffered solution in the range 2.9-4.1 similar to the formulation of insulin glargine in Lantus® (Sanofi, Paris, FR)) of a selected SCI-A and a selected SCI-B provides a biphasic insulin analogue formulation for the treatment of diabetes mellitus. This product may be used for twice a day injection or, in cultures in which people typically consume a single large meal per day, once a day injection. The latter therapeutic application would benefit from a basal component that is longer in duration than is characteristic of NPH microcrystalline suspensions and is more like insulin glargine (the active component of Lantus® and Toujeo®; Sanofi, Paris, FR) in its duration of action.

The premixed solution of two SCIs is intended to one or more of the properties: (i) resistance to degradation; (ii) substantial in vivo hypoglycemic potency; (iii) reduced cross-binding to IGF-1R; and (iv) biphasic pharmacokinetics and pharmacodynamics in the absence of a component consisting of a micro-crystalline suspension. The present invention provides a premixed solution of two SCIs—one with an isoelectric point in the range 4.5-6.0 and the other in the range 6.5-8.0—such that the two protein analogues are soluble and compatible within a single acidic solution and such that on subcutaneous injection both rapid-onset of action and a prolonged tail of action are achieved leading to an overall blood glucose control profile comparable to those of premixed products, such as those provided by “Humalog® Mix75/25” (Eli Lilly and Co., Indianapolis, Ind.) or “NovaMix® 30” (Novo Nordisk, Bagsvaerd, DK). Premixed biphasic SCI analog formulations of the present invention will therefore provide simplified once-a-day or twice-a-day bolus-basal regimens that will be of clinical advantage in the developed and developing world, especially for type 1 diabetes mellitus (T1DM). Single-chain biphasic insulin analogue formulations may also be initiated in insulin-naïve patients not well controlled on metformin, a first-line oral agent widely used in the treatment of type 2 diabetes mellitus (T2DM).

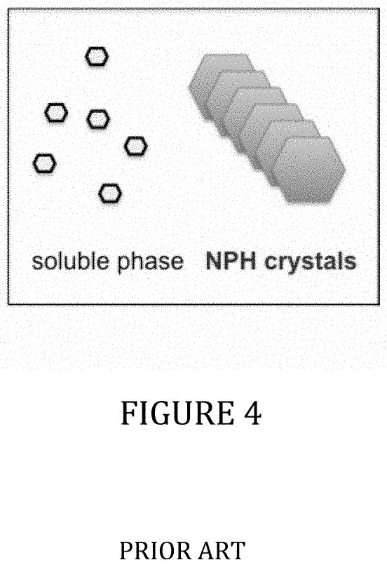

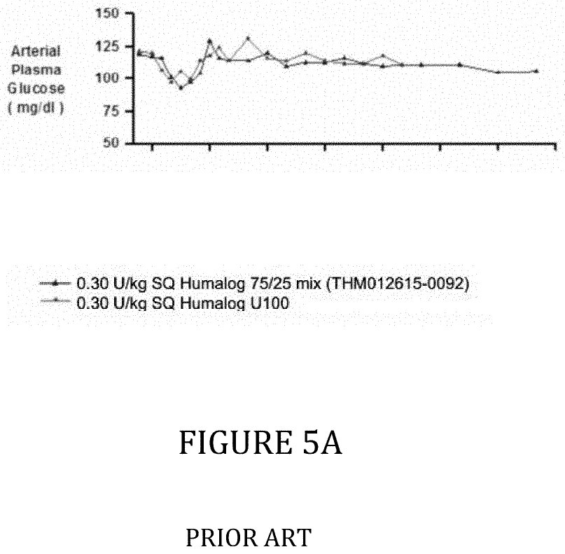

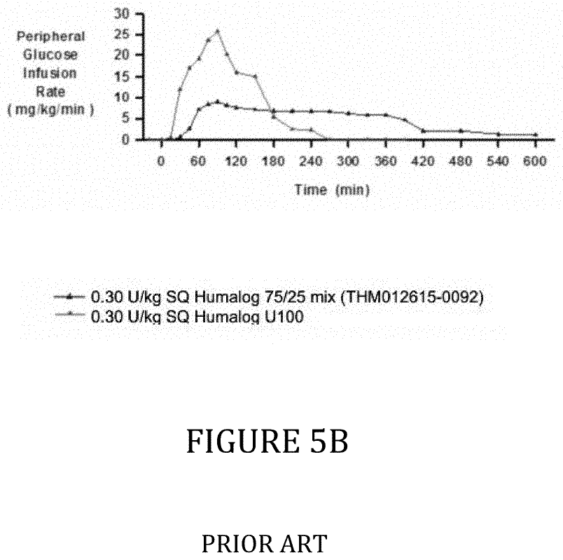

The mechanism of biphasic action of current regular-NPH premixed products, based on pharmacokinetic properties of the two components, is shown in schematic form in FIG. 4. These prior biphasic insulin products contain a soluble phase (single zinc hexamers) and an insoluble phase (associated NPH microcrystalline suspension). In this case two different states of matter, soluble and crystalline, distinguish between the fast-acting and long-acting components of current premixed products. Current FDA guidelines disallow mixing of Lantus® (Sanofi, Paris, FR; a pI-shifted long-acting insulin analogue formulated at pH 4.0) with rapid-acting insulin analogues formulated at or near neutral pH (e.g., Humalog® (Eli Lilly and Co., Indianapolis, Ind.), Novolog® (Novo Nordisk, Bagsvaerd, DK) or Apidra® (Sanofi, Paris, FR)). Such mixing is disallowed due to the risk of protein precipitation and due to the risk of altered PK/PD properties by the combined solution; i.e., that the mixed solutions can be perturbed in either or both their rapid-acting component (delayed in absorption by the basal analogue) and the basal component (accelerated in absorption by the rapid-acting analogue). It is therefore a surprising aspect of the present invention is that a single state of matter—a soluble mixture of a rapid-acting SCI and pI-shifted long-acting SCI in an acidic solution—can provide on subcutaneous injection of a single clear solution both a rapid-acting component and a basal component of insulin action. An example of the pharmacodynamic profile of a biphasic insulin product in current clinical use (Humalog® Mix75/25™; Eli Lilly and Co., Indianapolis, Ind.) is shown in FIG. 5 in relation to a related rapid-acting analog in current clinical use (Humalog® U-100; Eli Lilly and Co., Indianapolis, Ind.).

We envisage that the products of the present invention will particularly benefit patients in Western societies whose compliance with more complex regimens is uncertain. It is known in the art that health-care outcomes—and long-term adherence to prescribed regimens in chronic diseases such as T2DM and the metabolic syndrome—are a complex function of socioeconomic status, formal education, family structure, and cultural belief systems. Indeed, these societal issues are of increasing concern given the growing burden of obesity and T2DM in the United States and elsewhere among under-represented minorities, including African-Americans, Hispanic and indigenous Americans. Premixed single-chain insulin analogue formulations of the present invention will provide biphasic insulin action (rapid-acting and basal) and are therefore intended to benefit insulin-requiring T1DM and T2DM patients who have inadequate glycemic control with basal-only insulin therapy but for whom a full basal-bolus regimen is impractical or contraindicated by other medical factors.

The molar ratio of SCI-A:SCI-B may be selected depending on the particular requirements of a given patient. In one example, a 1:1 molar ratio may be used (similar to a current product containing insulin lispro partitioned 50% as NPH microcrystals and 50% as soluble zinc hexamers; Humalog® Mix50/50™, Eli Lilly and Co., Indianapolis, Ind.). In other examples, the formulation may be 80-65% SCI-A and 20-35% SCI-B (such that the sum of percentages is always 100%). In still another example, an acidic solution containing a molar SCI-A:SCI-B of 3:1 may be selected, similar to a current product containing insulin lispro partitioned 75% as NPH microcrystals and 25% as soluble zinc hexamers (Humalog® Mix75/25™, Eli Lilly and Co., Indianapolis, Ind.) at neutral pH. In yet another example, a 7:3 molar ratio of SCI-A to SCI-B may be selected, similar to Novolog® Mix70/30 which contains a 7:3 molar ratio of insulin aspart (AspB28-insulin) in NPH microcrystals and soluble zinc hexamers at neutral pH.

The present invention differs from such prior products in one or more of several important respects:
(i) The present SCI-A/SCI-B premixed solution exhibits more prolonged shelf life at or above room temperature, even at temperatures as extreme as 55° C.;
(ii) the present SCI-A/SCI-B premixed solution exhibits a flatter and more prolonged long-acting phase relative to NPH microcrystalline suspensions;
(iii) a soluble protein solution in a single phase may be more consistently dosed by a patient than can be a product containing a microcrystalline suspension;
(iv) the present SCI-A/SCI-B premixed solution is more readily tuneable in its pharmacokinetic properties by adjusting the molar ratio of zinc ions to protein component; and
(v) manufacture and coformulation of a soluble protein solution is less expensive than is the complex process by which soluble/NPH microcrystalline suspensions are typically prepared.

The mixture of two ultra-stable single-chain insulin analogues may provide biphasic absorption pharmacokinetics from a subcutaneous depot when co-formulated as a clear and soluble monophasic solution of the proteins at acidic pH (in the range 2.9-4.1). Conventional premixed products, as known in the art, represent an extreme end of a continuum of possible coupled equilibria between states of self-assembly, a soluble phase (single zinc hexamers) and an insoluble phase (associated NPH microcrystalline suspension; see FIG. 4). It is also possible that rapid- and delayed absorption characteristics can be retained from a subcutaneous depot formed by coinjection of SCI-A and SCI-B due to their different isoelectric points. In the present invention, inclusion of SCI-B in an acidic formulation of SCI-A provides a rapid-acting component whereas the basal component characteristic of SCI-A is retained. The latter is presumably due to the isoelectric precipitation of SCI-A in the subcutaneous space as the pH of the depot approaches neutrality. Although not wishing to be constrained by theory, we envision that most of the protein molecules of SCI-B “escape” trapping within the isoelectric precipate formed by SCI-A at neutral pH in the subcutaneous depot. This soluble fraction of SCI-B then provides the rapid-acting component.

A soluble co-formulation of SCI-A and SCI-B is feasible under acidic conditions (pH 2.9-4.1 in the absence of buffer) either (a) in the presence of 2-4 zinc ions per six protein monomers, inclusive of both SCIs; (b) in the presence of less than 2 zinc ions per six protein monomers such that hexamer assembly would be incomplete; or (c) even in the complete absence of zinc ions. Whereas zinc ions ordinarily contribute to the stability of an insulin formulation (or insulin analog formulation), the intrinsic stability of SCIs makes this contribution optional. Zinc-ion concentration can therefore be tuned with respect to optimal pharmacokinetic features.

It is also envisioned that single-chain analogues may also be made with A- and B-domain sequences derived from animal insulins, such as porcine, bovine, equine, and canine insulins, by way of non-limiting examples. In addition or in the alternative, the insulin analogue of the present invention may contain a deletion of residues B 1-B3 or may be combined with a variant B chain lacking Lysine (e.g., LysB29 in wild-type human insulin) to avoid Lys-directed proteolysis of a precursor polypeptide in yeast biosynthesis in Pichia pastoris, Saccharomyces cerevisciae, or other yeast expression species or strains. Furthermore, in view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine(Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Diaminobutyric acid, or Diaminopropionic acid), which may also be considered basic amino acids. Lys may also be replaced by the neutral aliphatic isostere Norleucine (Nle), which may in turn be substituted by analogues containing shorter aliphatic side chains (Aminobutyric acid or Aminopropionic acid).

The single-chain insulin analogues (SCIs) of the present invention have the general structure of an insulin B-chain polypeptide (B) linked to an insulin A-chain polypeptide (A) by a connecting or linker peptide (C) in the arrangement B-C-A. In either SCI, the connecting or linker peptides (C) may be any 5-11 amino acids. Unless stated or directly implied otherwise, positions of amino acids provided herein should be understood to the position relative to wild type polypeptides of insulin. For example, in the SCI-A provided as SEQ ID NO:5, having the arrangement of domains B-C-A as mentioned above, positions 1-30 of SEQ ID NO: 5 correspond to positions B1-B30 of wild type human insulin. Positions 31-38 of SEQ ID NO: 5 correspond to positions 1-8 of the C-domain or linker domain. Positions 39-59 of SEQ ID NO: 5 correspond to positions A1-A21 of wild type human insulin. One of skill in the art will recognize the same pattern for the arrangement of SEQ ID NOs: 6-10.

The A-domains may contain in each case may comprise a substitution at A21 (Gly, Ala, Gln or Ser) to avoid acid-catalyzed deamidation or other modes of Asn-related chemical degradation. The analogues of the present invention may also comprise Histidine at position B10 and so circumvent concerns regarding carcinogenesis that is associated with an acidic substitution (Aspartic Acid or Glutamic Acid) at this position. It is an additional aspect of the present invention that absolute in vitro affinities of the single-chain insulin analogue for IR-A and IR-B are in the range 5-100% relative to wild-type human insulin.

In one example, the C-domain design comprises a foreshortened connecting polypeptide (length 5-11 residues) containing an N-terminal acidic element (residues C1 and C2), a flexible joint or hinge (C3 and C4), and C-terminal segment containing a pair of basic residues analogous to those observed in natural proinsulins (C5 and C6). An upper limit of 11 for the C-domain length was chosen to be below the 12-residue size of an IGF-I-derived linker previously described in a chimeric insulin analogue which demonstrated enhanced IGF-1R-binding activity. A lower limit of 5 was chosen to preserve biological activity. In addition to their net effects on isoelectric point, the detailed positions of acidic and basic elements within the C domain can also confer favorable biological or biophysical properties. Although not wishing to be constrained by theory, we envision that the two-residue acidic residue introduces unfavorable electrostatic repulsion on binding of the analogue to IGF-1R but is well tolerated by insulin receptor isoforms. Also, without wishing to be constrained by theory, we further envision that the C-terminal basic motif contributes to partial subcutaneous aggregation rather than a mere tether or space element.

In one particular example, the connecting peptide (C domain) of the first SCI (SCI-A) may have the general sequence EXₙRRSR, where X is any amino acid and n=0-5. In one particular example, the C peptide of SCI-A is 8 amino acids long. In addition or in the alternative, the C peptide of SCI-A may have the sequence EEGSRRSR (positions 31-38 of SEQ ID NO: 5). Other C peptide sequences are also envisioned. In addition or in the alternative, the C peptide of SCI-B may have Glu on the N-terminal end and Arg, Ala or Ser on the C-terminal end, such that the C peptide may have any of the general sequences EXₙR, EXₙA or EXₙS, where X is any amino acid and n=3-9. In one example, SCI-B has a C peptide (or C domain) of 6 amino acids in length. In addition or in the alternative, the C peptide of SCI-B may have the sequence EEGPRR (positions 31-36 of SEQ ID NO: 6), EAAAAA (positions 31-36 of SEQ ID NO: 7), or EAAARA (positions 31-36 of SEQ ID NO: 8).

The A-domain of SCI-B may also contain a substitution at position A8 (Ala, Glu, Gln, His, Lys, or Arg); and a substitution at A14 (Glu or Ala).

The A-chain (or A-domain) polypeptides of SCI-A and/or SCI-B may comprise a substitution at the position corresponding to A21 of wild type insulin (the C-terminus of the SCIs) selected from Gly, Ala, Gln or Ser to avoid acid-catalyzed deamidation or other modes of Asn-related chemical degradation. The A-domain of SCI-A may also comprise a substitution of a basic amino acid at the position corresponding to position A8 of wild type insulin to raise its pI. Basic amino acids include Lysine, Arginine and Histidine, and also the non-standard amino acid Ornithine. The B-chain polypeptide (or B-domain) of SCI-A may comprise an Arg substitution at the position corresponding to B29 of wild type insulin to avoid Lys-specific proteolytic cleavage in the course of biosynthesis in yeast.

The A-domain of SCI-B may comprise a substitution at the position corresponding to A21 of wild type insulin (the C-terminus of the SCIs) selected from Gly, Ala, Gln or Ser as mentioned above, and may additionally comprise a non-Beta-branched amino acid at the position corresponding to A8 of wild type insulin, a substitution at the position corresponding to A14 of wild type insulin selected from Glu or Ala to avoid the reverse-hydrophobic effect presumably incurred by the wild-type TyrA14 and to provide an additional negative charge, or both substitutions at A8 and A14. In one particular example, the substitution at the position corresponding to A8 of wild type insulin may be a Glu, Ala Gln, His, Lys or Arg substitution to enhance stability and activity.

Additional examples of sequences of SCI-A are disclosed in U.S. Pat. No. 9,499,600, which is incorporated herein by reference. Examples of sequences of SCI-B are disclosed in U.S. Pat. No. 10,392,429, and in U.S. Pat. No. 8,192,957, which are likewise incorporated herein by reference.

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A chain of human insulin is provided as SEQ ID NO: 2.

(human A chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B chain of human insulin is provided as SEQ ID NO: 3.

(human B chain)
SEQ ID NO: 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of the modified B chain of KP-insulin (insulin lispro, the active component of Humalog®; Eli Lilly and Co., Indianapolis, Ind.) is provided as SEQ ID NO: 4.

(human “KP” B chain)
SEQ ID NO: 4
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Lys-Pro-Thr

The amino acid sequence of an exemplary SCI-A, possessing the substitutions ArgA8, GlyA21 and ArgB29 is provided as SEQ ID NO: 5.

(SCI-A)
SEQ ID NO: 5
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Arg-Thr-Glu-Glu-Gly-SerArg-Arg-
Ser-Arg-Gly-Ile-Val-Glu-Gln-Cys-Cys-Arg-Ser-Ile-
Cys-Ser-Leu-Tyr-Gln-Leu-Glu-Asn-Tyr-Cys-Gly

The amino acid sequence of an exemplary SCI-B, possessing the substitutions GluA14, GluB29 is provided as SEQ ID NO: 6.

SEQ ID NO: 6
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Glu-Thr-Glu-Glu-Gly-Pro-Arg-Arg-
Gly-Ile-Val-Glu-Gln-Cys-Cys-Thr-Ser-Ile-Cys-Ser-
Leu-Glu-Gln-Leu-Glu-Asn-Tyr-Cys-Asn

The amino acid sequence of an alternative SCI-B, possessing the substitutions GluA14, Asp B28, ProB29 is provided as SEQ ID NO: 7.

SEQ ID NO: 7
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Ala-Ala-Ala-Ala-Ala-
Gly-Ile-Val-Glu-Gln-Cys-Cys-Thr-Ser-Ile-Cys-Ser-
Leu-Glu-Gln-Leu-Glu-Asn-Tyr-Cys-Asn

The amino acid sequence of another alternative SCI-B, possessing the substitutions GluA14, Asp B28, ProB29 is provided as SEQ ID NO: 8.

SEQ ID NO: 8
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Ala-Ala-Ala-Arg-Ala-
Gly-Ile-Val-Glu-Gln-Cys-Cys-Thr-Ser-Ile-Cys-Ser-
Leu-Glu-Gln-Leu-Glu-Asn-Tyr-Cys-Asn

A generalized sequence for SCI-A may be provided as SEQ ID NO: 9.

SEQ ID NO: 9
Phe-Val-Asn-Gln-His-Leu-Cys-Gly-Ser-His-Leu-Val-
Glu-Xaa-Leu-Tyr-Leu-Val-Cys-Gly-Glu-Arg-Gly-Phe-
Phe-Tyr-Thr-Pro-Xaa-Thr-Xaa-Xaa-Xaa-Xaa-Xaa-Xaa-
Xaa-Xaa-Xaa-Xaa-Xaa-Gly-Ile-Val-Glu-Gln-Cys-Cys-
Xaa-Ser-Ile-Cys-Ser-Leu-Tyr-Gln-Leu-Glu-Asn-Tyr-
Cys-Xaa

Where the variable positions are defined as follows:
Position 14: Xara is Ala or Gln;
Position 29; Xaa is Arg or Lys;
Positions 31-41; Xaa is any 5-11 of amino acid;
Position 49: Xaa is Lys, Arg or His;
Position 62: Xaa is Gly, Ala, Ser, or Gln.

A generalized sequence for SCI-B may be provided as SEQ ID NO: 10.

SEQ ID NO: 10
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Xaa-Xaa-Thr-Xaa-Xaa-Xaa-Xaa-Xaa-Xaa-
Xaa-Xaa-Xaa-Xaa-Xaa-Gly-Ile-Val-Glu-Gln-Cys-Cys-
Xaa-Ser-Ile-Cys-Ser-Leu-Xaa-Gln-Leu-Glu-Asn-Tyr-
Cys-Asn

Where the variable positions are defined as follows:
Position 28: Xaa is Asp or Pro;
Position 29: Xaa is Lys, Pro or Glu;
Positions 31-41; Xaa is any 5-11 of amino acid;
Position 49: Xaa is any amino acid other than Leu, Be, or Val;
Position 55: Xaa is Glu, Ala or Tyr;
Position 62: Xaa is Asn, Gly, Ala, Ser or Gln.

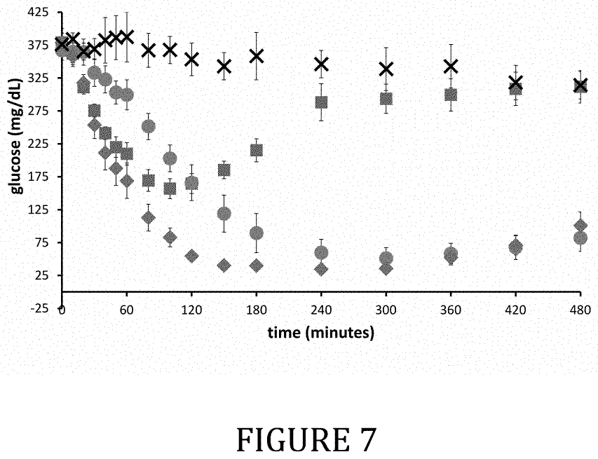

The principle that a rapid-acting insulin analogue may be premixed with a pI-shifted single-chain insulin analogue (SCI-A) in an unbuffered acidic solution to yield a soluble biphasic formulation was demonstrated through rat studies of Humalog® (Eli Lilly and Co., Indianapolis, Ind; as formulated by Lilly at neutral pH) in relation to a representative SCI-A or a 75:25 premixed solution of SCI-A or insulin lispro (the active component of Humalog®) as formulated at pH 4 like Lantus® (Sanofi, Paris FR). The SCI-A (SEQ ID NO: 5) contained an eight-residue C domain (sequence EEGSRRSR; positions 31-38 of SEQ ID NO: 5) and three substitutions with the insulin moiety (ArgA8, GlyA21 and ArgB29, all relative to the positions of wild type insulin). Biological activity and pharmacodynamics were tested in male Sprague-Dawley rats (ca. 300 g) rendered diabetic by streptozotocin (FIG. 7). The PD properties of the premixed formulation contained a fast-acting component and a basal component. The latter was greater in duration than that provided by an NPH microcrystalline suspension in this rat model.

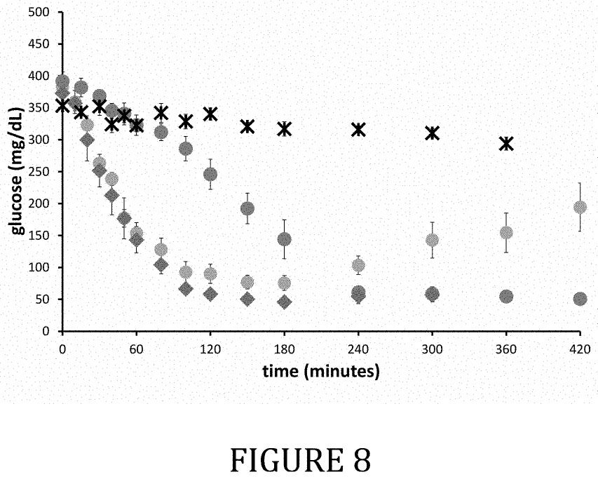

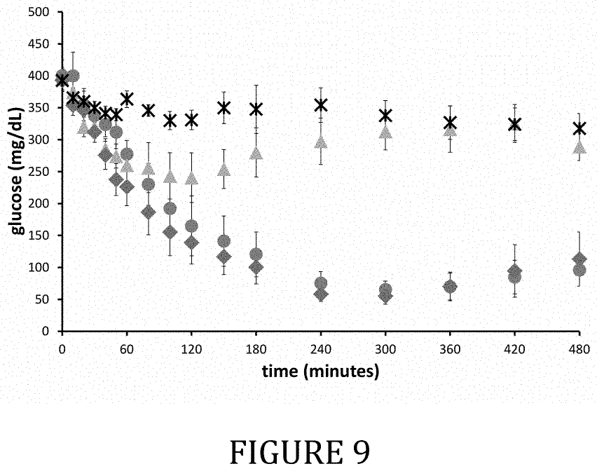

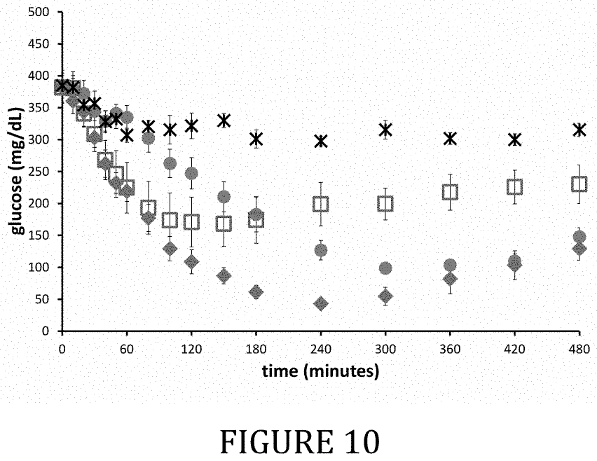

It was demonstrated that isoelectric precipitation of SCI-A would likewise not trap an SCI-B in the subcutaneous depot using three different embodiments of an SCI-B (FIGS. 8-10). Each of the three SCI-B analogues contained a six-residue C domain and an A-domain substitution known in the art to enhance stability and receptor-binding affinity (HisA8). The first SCI-B (SEQ ID NO: 6; FIG. 8) contained C domain sequence EEGPRR (positions 31-36). This analogue also contained insulin-moiety substitutions GluA14 and GluB29. The second example of an SCI-B (SEQ ID NO: 7; FIG. 9) contained C domain sequence EAAAAA (positions 31-36) and insulin-moiety substitutions GluA14, AspB28 and ProB29. The third example of an SCI-B (SEQ ID NO: 8; FIG. 10) contained C domain sequence EAAARA (positions 31-36) and also contained insulin-moiety substitutions GluA14, AspB28 and ProB29. In each example the 59-residue SCI-A (in a specific embodiment as defined above) did not blunt or prevent the premixed SCI-B component from directing a rapid initial phase of insulin action.

FIG. 8 provides blood glucose levels over time for diabetic rats (time 0 blood glucose of 350-400 [±20 mg/dl]) injected subcutaneously with 1 unit of the indicated analog/300 g body weight. ( [symbol] ) diluent control; (dark gray circles) an SCI-A (SEQ ID NO: 5); (light gray circles) an SCI-B (SEQ ID NO: 6) as formulated at neutral pH; and (♦) equimolar premixed clear solution of this SCI-A and this specific SCI-B as formulated in soluble form in an unbuffered solution at pH 4.0. Respective doses: 20 μg of each protein per 300 gram rat (singly); 20 μg and 20 μg in the case of the premixed solution to a total [SCI-A+SCI-B] combined dose of 40 μg.

FIG. 9 shows the blood glucose levels over time of diabetic rats (time 0 blood glucose of 400±20 mg/dl) injected subcutaneously with indicated analogue/300 g body weight. ( [symbol] ) diluent control; (●) an SCI-A (SEQ ID NO: 5); (▴) an SCI-B (SEQ ID NO: 7) as formulated at pH 4; and (♦) premixed clear solution of insulin SCI-A and SCI-B as formulated in soluble form (like Lantus®; Sanofi, Paris, FR) in an unbuffered solution at pH 4.0. Respective doses: 0.75 units SCI-A, 0.25 units SCI-B; and premixture of 0.75 units SCI-A and 0.25 units SCI-B.

FIG. 10 provides blood glucose levels over time of diabetic rats (time 0 blood glucose of 385±20 mg/dl) injected subcutaneously with indicated analogue/300 g body weight. ( [symbol] ) diluent control; (●) an SCI-A (SEQ ID NO: 5); ( [symbol] ) an SCI-B (SEQ ID NO: 8) as formulated at pH 4; and (♦) premixed clear solution of insulin SCI-A and SCI-B as formulated in soluble form (like Lantus®; Sanofi, Paris, FR) in an unbuffered solution at pH 4.0. Respective doses: 0.75 units SCI-A, 0.25 units SCI-B; and premixture of 0.75 units SCI-A and 0.25 units SCI-B.

It is envisioned that a polynucleotide sequence encoding SCI-A and/or SCI-B could be determined by one of skill in the art, given the well-known nature of codon usage. Such a polynucleotide could be a DNA or RNA sequence and codon usage could be determined according to typical usage in an organism of interest such as a yeast strain, such as Pichia pastoris, to maximize expression of the polynucleotide. Alternatively, it would be possible to prepare SCI-A and/or SCI-B by total chemical polypeptide synthesis.

A method for treating a patient with diabetes mellitus comprises administering a single-chain insulin analogue as described herein. We further envision the analogues of the present invention providing a method for the treatment of diabetes mellitus or the metabolic syndrome. The route of delivery of the insulin analogue is by subcutaneous injection through the use of a syringe or pen device.

A single-chain insulin analogue of the present invention may also contain other modifications, such as a halogen atom at positions B24, B25, or B26 as described more fully in U.S. Pat. No. 8,921,313, the disclosure of which is incorporated by reference herein. An insulin analogue of the present invention may also contain a foreshortened B-chain due to deletion of residues B1, B 1-B2 or B 1-B3 as described more fully in co-pending U.S. Provisional Patent Application 61/589,012.

A pharmaceutical composition may comprise such insulin analogues and which may optionally include zinc. In such a formulation, the concentration of the insulin analogue would typically be between about 0.6-5.0 mM; concentrations up to 5 mM may be used in vial or pen; the more concentrated formulations (U-200 or higher) may be of particular benefit in patients with marked insulin resistance. Excipients may include glycerol, glycine, arginine, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

Based upon the foregoing disclosure, it should now be apparent that a premixed solution of single-chain insulin analogues, as provided in this disclosure, will carry out the objects set forth hereinabove. Namely, that a premixed solution of SCI-A (representing a class of single-chain insulin analogues with isoelectric point between 6.5 and 8.0) and SCI-B (representing a class of single-chain insulin analogues with isoelectric point between 4.5 and 6.0) exhibit enhanced resistance to fibrillation while conferring desirable pharmacokinetic and pharmacodynamic features (conferring biphasic action) and maintaining at least a fraction of the biological activity of wild-type insulin. It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.

Brange J, editor. (1987) Galenics of Insulin: The Physico-chemical and Pharmaceutical Aspects of Insulin and Insulin Preparations. Berlin: Springer Berlin Heidelberg.

Glidden, M. D., Aldabbagh, K., Phillips., N. B., Can., K., Chen, Y. S., Whittaker, J., Phillips, M., Wickramasinghe, N. P., Rege, N., Swain, M., Peng, Y., Yang, Y., Lawrence, M. C., Yee,

V. C., Ismail-Beigi, F., & Weiss, M. A. 2017. An ultra-stable single-chain insulin analog resists thermal inactivation and exhibits biological signaling duration equivalent to the native protein. J. Biol. Chem. pii: jbc.M117.808626. doi: 10.1074/jbc.M117.808626 [Epub ahead of print]; in print: 293: 69-8

Glidden M D, Yang Y, Smith N A, Phillips N B, Carr K, Wickramasinghe N P, Ismail-Beigi F, Lawrence M C, Smith B J, Weiss M A. 2017. Solution structure of an ultra-stable single-chain insulin analog connects protein dynamics to a novel mechanism of receptor binding. J. Biol. Chem. pii: jbc.M117.808667. doi: 10.1074/jbc.M117.808667 [Epub ahead of print]; in print 293: 47-6

Hohsaka, T., and Sisido, M. (2012) Incorporation of non-natural amino acids into proteins. Curr. Opin. Chem. Biol. 6, 809-15.

Hua, Q. X., Nakagawa, S. H., Jia, W., Huang, K., Phillips, N. B., Hu, S. and Weiss, M. A. (2008) Design of an active ultrastable single-chain insulin analog: synthesis, structure, and therapeutic implications. J. Biol. Chem. 283, 14703-14716.

Ilag, L. L., Kerr, L., Malone, J. K., and Tan, M. H. (2007) Prandial premixed insulin analogue regimens versus basal insulin analogue regimens in the management of type 2 diabetes: an evidence-based comparison. Clin. Ther. 29, 1254-70.

Jang, H. C., Guler, S., and Shestakova, M. (2008) When glycaemic targets can no longer be achieved with basal insulin in type 2 diabetes, can simple intensification with a modern premixed insulin help? Results from a subanalysis of the PRESENT study. Int. J. Clin. Pract. 62, 1013-8.

Kalra, S., Balhara, Y., Sahay, B., Ganapathy, B., and Das, A. (2013) Why is premixed insulin the preferred insulin? Novel answers to a decade-old question. J. Assoc. Physicians India 61, 9-11.

Lee, H. C., Kim, S. J., Kim, K. S., Shin, H. C., and Yoon, J. W. (2000) Remission in models of type 1 diabetes by gene therapy using a single-chain insulin analogue. Nature 408, 483-8. Retraction in:Lee H C, Kim K S, Shin H C. 2009. Nature 458, 600.

Mosenzon, O., and Raz, I. (2013) Intensification of insulin therapy for type 2 diabetic patients in primary care: basal-bolus regimen versus premix insulin analogs: when and for whom? Diabetes Care 36 Suppl 2, S212-8.

Phillips, N. B., Whittaker, J., Ismail-Beigi, F., and Weiss, M. A. (2012) Insulin fibrillation and protein design: topological resistance of single-chain analogues to thermal degradation with application to a pump reservoir. J. Diabetes Sci. Technol. 6, 277-288.

Qayyum, R., Bolen, S., Maruthur, N., Feldman, L., Wilson, L. M., Marinopoulos, S. S., et al. (2008) Systematic review: comparative effectiveness and safety of premixed insulin analogues in type 2 diabetes. Ann. Intern. Med. 149, 549-59.

Rolla, A. R., and Rakel, R. E. (2005) Practical approaches to insulin therapy for type 2 diabetes mellitus with premixed insulin analogues.Clin. Ther. 27, 1113-25.

Wang, Z. X. (1995) An exact mathematical expression for describing competitive biding of two different ligands to a protein molecule FEBS Lett. 360: 111-114.

Whittaker, J., and Whittaker, L. (2005) Characterization of the functional insulin binding epitopes of the full-length insulin receptor. J. Biol. Chem. 280: 20932-20936.

What is claimed is:

1. A pharmaceutical formulation comprising a premixed clear mixture of a first single-chain insulin analogue and a second single-chain insulin analogue, wherein the first and second single-chain insulin analogues comprise the structure B-C-A, where B is an insulin B-chain polypeptide, where A is an insulin A-chain polypeptide and wherein C is a connecting polypeptide of 5-11 amino acids in length, wherein the first insulin analogue has an isoelectric point between 6.5 and 8.0 and where the second insulin analogue has an isoelectric point of between 4.5 and 6.0.

2. The pharmaceutical formulation of claim 1, wherein the pH of the formulation is between pH 2.9 and pH 4.1.

3. The pharmaceutical formulation of claim 1, wherein at least one of the first and second single-chain insulin analogues comprises a Gly, Ala, Ser or Gln substitution at the position corresponding to position A21, relative to wild type insulin.

4. The pharmaceutical formulation of claim 3, wherein the first single-chain insulin analogue comprises a Lys, Arg or His substitution at the position corresponding to position A8 relative to wild type insulin and optionally comprises an Arg substitution at the position corresponding to position B29 relative to wild type insulin.

5. The pharmaceutical formulation of claim 4, where the first single-chain insulin analogue additionally comprises a Gln substitution at the position corresponding to position B13 relative to wild type insulin.

6. The pharmaceutical formulation of claim 4, wherein the first single-chain insulin analogue comprises a C domain having the sequence EXₙRRSR where X is any amino acid and n is 0-6.

7. The pharmaceutical formulation of claim 6, wherein the first single-chain insulin analogue comprises SEQ ID NO: 5.

8. The pharmaceutical formulation of claim 4, wherein the second single-chain insulin analogue comprises one or more substitutions, relative to the positions of wild type insulin, selected from the group consisting of:
a non-beta-branched residue at position A8,
a Glu or Ala at position A14,
a Glu or Lys substitution at position B28, and
a Pro or Glu substitution at position B29.

9. The pharmaceutical formulation of claim 8, wherein the second single-chain insulin analogue comprises a Glu, Ala, Gln, His, Lys or Arg substitution at the position corresponding to A8 of wild type insulin.

10. The pharmaceutical formulation of claim 9, where the second single-chain insulin analogue contains a C domain comprising the sequence EXₙR, EXₙA or EXₙS where n represents 3-9 of any amino acid.

11. The pharmaceutical formulation of claim 10, wherein the second single-chain insulin analogue comprises a sequence selected from the group consisting of SEQ ID NOs: 6-8.

12. The pharmaceutical formulation of claim 8, comprising an equimolar ratio of the first single-chain insulin analogue and the second single-chain insulin analogue.

13. The pharmaceutical formulation of claim 8, comprising a molar concentration of 65-80% of the first single-chain insulin analogue and 35-20% of the second single-chain insulin analogue such that the relative percentages of the two components totals 100%.

14. A method of treating a patient comprising administering a physiologically effective amount of a pharmaceutical formulation, wherein the pharmaceutical composition comprises a premixed clear mixture of a first single-chain insulin analogue and a second single-chain insulin analogue, wherein the first and second single-chain insulin analogues comprise the structure B-C-A, where B is an insulin B-chain polypeptide, where A is an insulin A-chain polypeptide and wherein C is a connecting polypeptide of 5-11 amino acids in length, wherein the first insulin analogue has an isoelectric point between 6.5 and 8.0 and where the second insulin analogue has an isoelectric point of between 4.5 and 6.0.

15. The pharmaceutical formulation of claim 1 wherein the formulation additionally contains zinc ions at a molar ratio of between 2 and 4 zinc ions per six single-chain insulin analogue monomers, inclusive of both proteins.

16. The pharmaceutical formulation of claim 1 wherein the formulation contains fewer than 2 zinc ions per six single-chain insulin analogue monomers inclusive of both proteins.

17. (canceled)

18. (canceled)

19. The method of claim 14, wherein at least one of the first and second single-chain insulin analogues comprises a Gly, Ala, Ser or Gln substitution at the position corresponding to position A21, relative to wild type insulin.

20. The method of claim 19, wherein the first single-chain insulin analogue comprises a Lys, Arg or His substitution at the position corresponding to position A8 relative to wild type insulin and optionally comprises an Arg substitution at the position corresponding to position B29 relative to wild type insulin.

21. The pharmaceutical formulation of claim 1, where the first single-chain insulin analogue additionally comprises a Gln substitution at the position corresponding to position B13 relative to wild type insulin.

22. The pharmaceutical formulation of claim 1, wherein the first single-chain insulin analogue comprises a C domain having the sequence EXₙRRSR where X is any amino acid and n is 0-6.
